SEQUENCE LISTING (9 sequences)

SEQ ID NO: 1 - DNA, 2829 bp, Homo sapiens
  source 1..2829: mol_type = genomic DNA
atgctcctgctgctggggctgtgcctggggctgtccctgtgtgtggggtcgcaggaagaggcgcagagctggggccactcttcggagcaggatggactcagggtcccgaggcaagtcagactgttgcagaggctgaaaaccaaacctttgatgacagaattctcagtgaagtctaccatcatttcccgttatgccttcactacggtttcctgcagaatgctgaacagagcttctgaagaccaggacattgagttccagatgcagattccagctgcagctttcatcaccaacttcactatgcttattggagacaaggtgtatcagggcgaaattacagagagagaaaagaagagtggtgatagggtaaaagagaaaaggaataaaaccacagaagaaaatggagagaaggggactgaaatattcagagcttctgcagtgattcccagcaaggacaaagccgcctttttcctgagttatgaggagcttctgcagaggcgcctgggcaagtacgagcacagcatcagcgtgcggccccagcagctgtccgggaggctgagcgtggacgtgaatatcctggagagcgcgggcatcgcatccctggaggtgctgccgcttcacaacagcaggcagaggggcagtgggcgcggggaagatgattctgggcctcccccatctactgtcattaaccaaaatgaaacatttgccaacataatttttaaacctactgtagtacaacaagccaggattgcccagaatggaattttgggagactttatcattagatatgacgtcaatagagaacagagcattggggacatccaggttctaaatggctattttgtgcactactttgctcctaaagaccttcctcctttacccaagaatgtggtattcgtgcttgacagcagtgcttctatggtgggaaccaaactccggcagaccaaggatgccctcttcacaattctccatgacctccgaccccaggaccgtttcagtatcattggattttccaaccggatcaaagtatggaaggaccacttgatatcagtcactccagacagcatcagggatgggaaagtgtacattcaccatatgtcacccactggaggcacagacatcaacggggccctgcagagggccatcaggctcctcaacaagtacgtggcccacagtggcattggagaccggagcgtgtccctcatcgtcttcctgacggatgggaagcccacggtcggggagacgcacaccctcaagatcctcaacaacacccgagaggccgcccgaggccaagtctgcatcttcaccattggcatcggcaacgacgtggacttcaggctgctggagaaactgtcgctggagaactgtggcctcacacggcgcgtgcacgaggaggaggacgcaggctcgcagctcatcgggttctacgatgaaatcaggaccccgctcctctctgacatccgcatcgattatccccccagctcagtggtgcaggccaccaagaccctgttccccaactacttcaacggctcggagatcatcattgcggggaagctggtggacaggaagctggatcacctgcacgtggaggtcaccgccagcaacagtaagaaattcatcatcctgaagacagatgtgcctgtgcggcctcagaaggcagggaaagatgtcacaggaagccccaggcctggaggcgatggagagggggacaccaaccacatcgagcgtctctggagctacctcaccacaaaggagctgctgagctcctggctgcaaagtgacgatgaaccggagaaggagcggctgcggcagcgggcccaggccctggctgtgagctaccgcttcctcactcccttcacctccatgaagctgagggggccggtcccacgcatggatggcctggaggaggcccacggcatgtcggctgccatgggacccgaaccggtggtgcagagcgtgcgaggagctggcacgcagccaggacctttgctcaagaagccataccagccaagaattaaaatctctaaaacatcagtggatggtgatccccactttgttgtggatttccccctgagcagactcaccgtgtgcttcaacattgatgggcagcccggggacatcctcaggctggtctctgatcacagggactctggtgtcacagtgaacggagagttaattggggcacccgcccctccaaatggccacaagaaacagcgcacttacttgcgcactatcaccatcctcatcaacaagccagagagatcttatctcgagatcacaccgagcagagtcatcttggatggtggggacagactggtgctcccctgcaaccagagtgtggtggtggggagctgggggctggaggtgtccgtgtctgccaacgccaatgtcaccgtcaccatccagggctccatagcctttgtcatcctcatccacctctacaaaaagccggcgcccttccagcgacaccacctgggtttctacattgccaacagcgagggcctttccagcaactgccacggactgctgggtcagttcctgaatcaggatgccagactcacagaagaccctgcagggcccagccagaacctcactcaccctctgctccttcaggtgggagaggggcctgaggccgtcctaacagtgaaaggccaccaagtcccagtggtctggaagcaaaggaagatttacaacggggaagagcagatagactgctggtttgccaggaacaatgccgccaaactgattgacggggagtacaaggattacctggcatcccatccatttgacacagggatgacacttggccggggaatgtccagggagctctga

SEQ ID NO: 2 - protein, 942 aa, Homo sapiens
MLLLLGLCLGLSLCVGSQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 3 - protein, 926 aa, Homo sapiens
SQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 4 - protein, 127 aa, Homo sapiens
VPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEE

SEQ ID NO: 5 - protein, 184 aa, Homo sapiens
NVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEI

SEQ ID NO: 6 - protein, 728 aa, Homo sapiens
MRNYDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGDPHFVVDFPLSRLTVCFNIDGQPGDILRLVSDHRDSGVTVNGELIGAPAPPNGHKKQRTYLRTITILINKPERSYLEITPSRVILDGGDRLVLPCNQSVVVGSWGLEVSVSANANVTVTIQGSIAFVILIHLYKKPAPFQRHHLGFYIANSEGLSSNCHGLLGQFLNQDARLTEDPAGPSQNLTHPLLLQVGEGPEAVLTVKGHQVPVVWKQRKIYNGEEQIDCWFARNNAAKLIDGEYKDYLASHPFDTGMTLGRGMSREL

SEQ ID NO: 7 - protein, 702 aa, Homo sapiens
MLLLLGLCLGLSLCVGSQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVRGFRREEQIFSNVMKFPFASILEL

SEQ ID NO: 8 - protein, 665 aa, Homo sapiens
SQEEAQSWGHSSEQDGLRVPRQVRLLQRLKTKPLMTEFSVKSTIISRYAFTTVSCRMLNRASEDQDIEFQMQIPAAAFITNFTMLIGDKVYQGEITEREKKSGDRVKEKRNKTTEENGEKGTEIFRASAVIPSKDKAAFFLSYEELLQRRLGKYEHSISVRPQQLSGRLSVDVNILESAGIASLEVLPLHNSRQRGSGRGEDDSGPPPSTVINQNETFANIIFKPTVVQQARIAQNGILGDFIIRYDVNREQSIGDIQVLNGYFVHYFAPKDLPPLPKNVVFVLDSSASMVGTKLRQTKDALFTILHDLRPQDRFSIIGFSNRIKVWKDHLISVTPDSIRDGKVYIHHMSPTGGTDINGALQRAIRLLNKYVAHSGIGDRSVSLIVFLTDGKPTVGETHTLKILNNTREAARGQVCIFTIGIGNDVDFRLLEKLSLENCGLTRRVHEEEDAGSQLIGFYDEIRTPLLSDIRIDYPPSSVVQATKTLFPNYFNGSEIIIAGKLVDRKLDHLHVEVTASNSKKFIILKTDVPVRPQKAGKDVTGSPRPGGDGEGDTNHIERLWSYLTTKELLSSWLQSDDEPEKERLRQRAQALAVSYRFLTPFTSMKLRGPVPRMDGLEEAHGMSAAMGPEPVVQSVRGAGTQPGPLLKKPYQPRIKISKTSVDGD

SEQ ID NO: 9 - DNA, 2829 bp, Homo sapiens
  source 1..2829: mol_type = other DNA
tcagagctccctggacattccccggccaagtgtcatccctgtgtcaaatggatgggatgccaggtaatccttgtactccccgtcaatcagtttggcggcattgttcctggcaaaccagcagtctatctgctcttccccgttgtaaatcttcctttgcttccagaccactgggacttggtggcctttcactgttaggacggcctcaggcccctctcccacctgaaggagcagagggtgagtgaggttctggctgggccctgcagggtcttctgtgagtctggcatcctgattcaggaactgacccagcagtccgtggcagttgctggaaaggccctcgctgttggcaatgtagaaacccaggtggtgtcgctggaagggcgccggctttttgtagaggtggatgaggatgacaaaggctatggagccctggatggtgacggtgacattggcgttggcagacacggacacctccagcccccagctccccaccaccacactctggttgcaggggagcaccagtctgtccccaccatccaagatgactctgctcggtgtgatctcgagataagatctctctggcttgttgatgaggatggtgatagtgcgcaagtaagtgcgctgtttcttgtggccatttggaggggcgggtgccccaattaactctccgttcactgtgacaccagagtccctgtgatcagagaccagcctgaggatgtccccgggctgcccatcaatgttgaagcacacggtgagtctgctcagggggaaatccacaacaaagtggggatcaccatccactgatgttttagagattttaattcttggctggtatggcttcttgagcaaaggtcctggctgcgtgccagctcctcgcacgctctgcaccaccggttcgggtcccatggcagccgacatgccgtgggcctcctccaggccatccatgcgtgggaccggccccctcagcttcatggaggtgaagggagtgaggaagcggtagctcacagccagggcctgggcccgctgccgcagccgctccttctccggttcatcgtcactttgcagccaggagctcagcagctcctttgtggtgaggtagctccagagacgctcgatgtggttggtgtccccctctccatcgcctccaggcctggggcttcctgtgacatctttccctgccttctgaggccgcacaggcacatctgtcttcaggatgatgaatttcttactgttgctggcggtgacctccacgtgcaggtgatccagcttcctgtccaccagcttccccgcaatgatgatctccgagccgttgaagtagttggggaacagggtcttggtggcctgcaccactgagctggggggataatcgatgcggatgtcagagaggagcggggtcctgatttcatcgtagaacccgatgagctgcgagcctgcgtcctcctcctcgtgcacgcgccgtgtgaggccacagttctccagcgacagtttctccagcagcctgaagtccacgtcgttgccgatgccaatggtgaagatgcagacttggcctcgggcggcctctcgggtgttgttgaggatcttgagggtgtgcgtctccccgaccgtgggcttcccatccgtcaggaagacgatgagggacacgctccggtctccaatgccactgtgggccacgtacttgttgaggagcctgatggccctctgcagggccccgttgatgtctgtgcctccagtgggtgacatatggtgaatgtacactttcccatccctgatgctgtctggagtgactgatatcaagtggtccttccatactttgatccggttggaaaatccaatgatactgaaacggtcctggggtcggaggtcatggagaattgtgaagagggcatccttggtctgccggagtttggttcccaccatagaagcactgctgtcaagcacgaataccacattcttgggtaaaggaggaaggtctttaggagcaaagtagtgcacaaaatagccatttagaacctggatgtccccaatgctctgttctctattgacgtcatatctaatgataaagtctcccaaaattccattctgggcaatcctggcttgttgtactacagtaggtttaaaaattatgttggcaaatgtttcattttggttaatgacagtagatgggggaggcccagaatcatcttccccgcgcccactgcccctctgcctgctgttgtgaagcggcagcacctccagggatgcgatgcccgcgctctccaggatattcacgtccacgctcagcctcccggacagctgctggggccgcacgctgatgctgtgctcgtacttgcccaggcgcctctgcagaagctcctcataactcaggaaaaaggcggctttgtccttgctgggaatcactgcagaagctctgaatatttcagtccccttctctccattttcttctgtggttttattccttttctcttttaccctatcaccactcttcttttctctctctgtaatttcgccctgatacaccttgtctccaataagcatagtgaagttggtgatgaaagctgcagctggaatctgcatctggaactcaatgtcctggtcttcagaagctctgttcagcattctgcaggaaaccgtagtgaaggcataacgggaaatgatggtagacttcactgagaattctgtcatcaaaggtttggttttcagcctctgcaacagtctgacttgcctcgggaccctgagtccatcctgctccgaagagtggccccagctctgcgcctcttcctgcgaccccacacacagggacagccccaggcacagccccagcagcaggagcat

(19) United States
(12) Patent Application Publication
(10) Pub. No.: US 2026/0199430 A1
(43) Pub. Date: Jul. 16, 2026

(54) ITIH5 FOR USE IN THE TREATMENT OF OBESITY

(71) Applicant: Rheinisch-Westfälische Technische Hochschule Aachen, Körperschaft Des Öffentlichen Rechts, Aachen (DE)

(72) Inventors: Edgar Dahl, Aachen (DE); Tim Ruhl, Klinik (DE); Sophia Villwock, Aachen (DE); Jana Keimes, Aachen (DE)

(21) Appl. No.: 19/133,906

(22) PCT Filed: Nov. 28, 2023
(86) PCT No.: PCT/EP2023/083244

§ 371 Date: May 29, 2025

(30) Foreign Application Priority Data
Nov. 29, 2022 (DE) 10 2022 131 546.0

(51) Int. Cl. A61K 38/17; A61P 3/04; G01N 33/50; G01N 33/68

(52) U.S. Cl. CPC A61K 38/1709; A61P 3/04; G01N 33/5073; G01N 33/6878

(57) ABSTRACT

The invention relates to an isolated or purified ITIH5 polypeptide having the biological activity of a negative regulator of adipogenesis for use in the treatment of obesity in a subject. The invention relates further to methods for identifying ITIH5 epitopes that are effective in reducing weight or weight gain and therefore constitute the basis for identifying synthetic peptides for use in the treatment of obesity. The invention also relates to a method for identifying small molecules that reduce weight or weight gain of a subject by modulation of the ITIH5-adipogenic pathway.

### FIELD OF THE INVENTION

The invention relates to an isolated or purified ITIH5 polypeptide having the biological activity of a negative regulator of adipogenesis for use in the treatment of obesity in a subject. The invention relates further to methods for identifying ITIH5 epitopes that are effective in reducing weight or weight gain and therefore constitute the basis for identifying synthetic peptides for use in the treatment of obesity. The invention also relates to a method for identifying small molecules that reduce weight or weight gain of a subject by modulation of the ITIH5-adipogenic pathway.

### BACKGROUND OF THE INVENTION

Obesity and its associated disorders are common and very serious public health problems in the United States and throughout the world. In the past two decades, obesity rates in the U.S. have increased from 30.5 percent of adults in 1999 to 41.9 percent of adults in 2020. Severe obesity has almost doubled from 4.7 to 9.2 percent, according to the U.S. Centers for Disease Control and Prevention (CDC). Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. It reduces lifespan and carries a serious risk of co-morbidities above, as well disorders such as inflammation, infections, varicose veins, acanthosis nigricans, eczema, exercise intolerance, insulin resistance, hypertension, hypercholesterolemia, cholelithiasis, orthopedic injury, and thromboembolic disease. Obesity is also a risk factor for the group of conditions called insulin resistance syndrome, or “Syndrome X”.

Recent estimates for the medical therapeutic cost of obesity and associated disorders are $150 billion worldwide. The pathogenesis of obesity is believed to be multifactorial, but the basic problem is that in obese subjects' nutrient availability and energy expenditure are disbalanced until there is excess of adipose tissue. Obesity is currently a poorly treatable, chronic, essentially intractable disease. A therapeutic drug useful in weight reduction of obese persons could have a profound beneficial effect on their health.

The Inter-alpha-Trypsin Inhibitor Heavy chain 5 (ITIH5) is a heavy chain component of one of the inter-alpha-trypsin inhibitor (ITI) family members. ITIs are composed of a light chain, called bikunin that confers a protease-inhibitor function, and five so far described different homologous heavy chains (ITIH1-5). ITIH5 was identified in 2004 as a new member of the ITI gene family of extracellular matrix proteins (HIMMELFARB et al., “ITIH5, a novel member of the inter-alpha-trypsin inhibitor heavy chain family is downregulated in breast cancer”, Cancer Letters, February 2004, Vol. 204 No. 1, pages 69-77). ITIH5 is thought to function as a tumor suppressor in several solid tumors including breast and thyroid cancers.

Later on, it was found out that ITIH5 interacts with hyaluronic acid (HA), an extracellular glycosaminoglycan that is dynamically produced by most skin cells, primarily fibroblasts in the dermis and keratinocytes in the epidermis. By interacting with HA, ITIH5 was shown to trigger biological processes in skin including ECM stabilization and epidermal differentiation and on the other hand modulating inflammatory responses. As a result, it was discussed as a therapeutic option for patients experiencing epidermal barrier dysfunction or UV light-induced skin damage, where ITIH5 as a natural stabilizer of HA could counteract these conditions (RUTH et al., “Inter-α-Trypsin Inhibitor Heavy Chain 5 (ITIH5) Is a Natural Stabilizer of Hyaluronan That Modulates Biological Processes in the Skin”, Skin Pharmacology and Physiology, October 2020, Vol. 33, No. 4, pages 198 to 206).

In 2012, Anveden et al. observed high expression of ITIH5 in adipose tissue which was found to be increased in obesity and down regulated after a hypocaloric diet-induced weight loss (ANVEDEN et al., “ITIH-5 Expression in Human Adipose Tissue Is Increased in Obesity”, Obesity, September 2012, Vol. 20, pages 708-714). These findings suggest that ITIH5 appears to be a positive regulator of obesity or at least a biomarker indicative of obesity.

Hence, there is still a need for the efficient and safe treatment and prevention of obesity and for treating conditions related to or caused by obesity, and for methods to identify compounds useful in such methods.

The objective of the present invention thus is to provide an improvement or an alternative to the prior art.

This problem is solved by provision of an ITIH5 polypeptide for use in the treatment of obesity according to claim 1, and methods for identifying small synthetic peptides or ITIH5-mimetic small molecules that reduce weight or weight gain of a subject according to claims 10 to 13. Specific embodiments are subject matter of further dependent claims.

### SUMMARY OF THE INVENTION

In a first aspect the present invention provides an isolated or purified ITIH5 polypeptide having the biological activity of a negative regulator of adipose stem cell proliferation and development into mature adipocytes for use in the treatment of obesity in a subject, wherein said treatment comprises reducing weight or reducing weight gain, wherein the ITIH5 polypeptide is to be administered to said subject in an amount therapeutically effective to reduce weight or weight gain, and wherein said ITIH5 polypeptide comprises or consists of a peptide which is selected from the group consisting of:
a) SEQ ID NO:2, SEQ ID NO:3, SEQ ID NO:4, SEQ ID NO:5, SEQ ID NO:6, SEQ ID NO:7 and SEQ ID NO:8,
b) a biologically active fragment, analog, variant, derivative or elongation of a),
c) a biologically active fragment, analog, variant or derivative of a) having at least 90% sequence identity to SEQ ID NO:2, SEQ ID NO:3, SEQ ID NO:4, SEQ ID NO:5, SEQ ID NO:6, SEQ ID NO:7 or SEQ ID NO:8,
d) a polypeptide encoded by the polynucleotide set out in SEQ ID NO:1,
e) a biologically fragment, analog, variant, derivative or elongation of c),
f) a polypeptide encoded by a polynucleotide that hybridizes to the polynucleotide set out in SEQ ID NO:9 under moderately stringent hybridization conditions.

The invention is based on the surprising finding, that ITIH5 is not only a protein expressed in adipose tissue but when given as a recombinant protein externally to human adipose stem cells (ASCs), ITIH5 is able to (i) reduce the proliferation of ASCs and also (ii) reduce the differentiation of ASCs into adipocytes. In both tests a dose-dependent effect was shown: The higher the ITIH5-dose the higher the inhibition of ASC proliferation and differentiation.

In sum, ITIH5 has to be regarded as a multi-effective negative regulator of adipogenesis and thus the ITIH5 protein or a fragment thereof or a ITIH5-mimicking small molecule compound qualify for use in a treatment or prevention of obesity.

### DETAILED DESCRIPTION OF THE INVENTION

The present invention is based on the finding of the inventors that an isolated ITIH5 polypeptide shows a twofold biological effect on human ASCs, making it to a negative regulator of adipogenesis, which accordingly qualify it for a use in the treatment of obesity.

These human in vitro findings are supported by the phenotype of the ITIH5 k.o. mouse showing an increased absolute fat weight and also an increased relative fat weight as compared to wildtype mice. Furthermore, in vitro-cultured ASCs from ITIH5^{-/-} mice show increased proliferation and increased adipogenic differentiation as compared to wildtype ASCs.

According to the present disclosure, the ITIH5 polypeptide of the present invention is a protein that contains or consists of amino acid position 1 to 942 of the sequence of human isoform 1 as depicted in SEQ ID NO:2. In an advantageous embodiment, the ITIH5 polypeptide consists of the amino acid of SEQ ID NO:2 and thus has a length of 942 amino acids.

In a further aspect, the ITIH5 polypeptide of the present invention is a protein that contains or consists of amino acid position 17 to 942 of the sequence of human isoform 1 as depicted in SEQ ID NO:3, thereby representing the mature ITIH5 protein without the signal peptide AS 1 to 16. In an advantageous embodiment, the ITIH5 polypeptide consists of the amino acid of SEQ ID NO:3 and thus has a length of 926 amino acids.

In another aspect, the ITIH5 polypeptide of the present invention is a protein that contains or consists of amino acid position 35 to 161 of the sequence of human isoform 1 as depicted in SEQ ID NO:4, thereby representing the VIT domain of the ITIH5 protein. The vault protein inter-alpha-trypsin (VIT) domain described here is found to the N-terminus of a von Willebrand factor type A domain (PF00092) in ITI heavy chains (ITIHs) and their precursors (HIMMELFARB et al. 2004, Cancer Letters, 204(1): 69-77). In an advantageous embodiment, the VIT-domain consists of the amino acid of SEQ ID NO:4 and thus has a length of 127 amino acids.

In one aspect, the ITIH5 polypeptide of the present invention is a protein that contains or consists of amino acid position 295 to 478 of the sequence of human isoform 1 as depicted in SEQ ID NO:5, thereby representing the vWFA domain of the ITIH5 protein. The von Willebrand factor is a large multimeric glycoprotein found in blood plasma. Mutant forms are involved in the aetiology of bleeding disorders. In von Willebrand factor, the type A domain (vWFA) is the prototype for a protein superfamily. The vWFA domain is found in various plasma proteins: complement factors B, C2, CR3 and CR4; the integrins (I-domains); collagen types VI, VII, XII and XIV; and other extracellular proteins. Although the majority of vWFA-containing proteins are extracellular, the most ancient ones present in all eukaryotes are all intracellular proteins involved in functions such as transcription, DNA repair, ribosomal and membrane transport and the proteasome (Ruggeri Z M & Ware J, 1993, “von Willebrand factor”, FASEB J. 7(2): 308-316. PMID 8440408). In an advantageous embodiment, the vWFA-domain consists of the amino acid of SEQ ID NO:5 and thus has a length of 184 amino acids.

In a further aspect, the ITIH5 polypeptide of the present invention is a protein that contains or consists of amino acid position 1 to 728 of the sequence as depicted in SEQ ID NO:6, thereby representing the isoform 2 of the human ITIH5 protein. In an advantageous embodiment, the ITIH5 polypeptide consists of the amino acid of SEQ ID NO:6 and thus has a length of 728 amino acids.

In another aspect, the ITIH5 polypeptide of the present invention is a protein that contains or consists of amino acid position 1 to 702 of the sequence as depicted in SEQ ID NO:7, thereby representing the isoform 3 of the human ITIH5 protein. In an advantageous embodiment, the ITIH5 polypeptide consists of the amino acid of SEQ ID NO:7 and thus has a length of 702 amino acids.

In another aspect, the ITIH5 polypeptide of the present invention is a protein that contains or consists of amino acid position 17 to 681 of the sequence as depicted in SEQ ID NO:8, thereby representing the mature human ITIH5 protein with a sequence N-terminal to the conserved cleavage site “DPHFVV” of ITIH proteins. In an advantageous embodiment, the ITIH5 polypeptide consists of the amino acid of SEQ ID NO:8 and thus has a length of 665 amino acids.

In a further aspect of the invention, the ITIH5 polypeptide is a biologically active fragment, analog, variant, derivative or elongation of the polypeptides of SEQ ID NO:2, SEQ ID NO:3, SEQ ID NO:4, SEQ ID NO:5, SEQ ID NO:6, SEQ ID NO:7 or SEQ ID NO:8 having at least 90% sequence identity to one of the aforementioned polypeptide sequences NO:2 to 8.

In one aspect of the invention, the ITIH5 polypeptide is a polypeptide encoded by the polynucleotide set out in SEQ ID NO:1, representing the consensus coding sequence of the human ITIH5 isoform 1 transcript and stored under the identifier CCDS31139.2 in the CCDS database (CCDS Release 24—Update for Human, Oct. 26, 2022). Notably, due to the possibility of a recoding in certain species, the polypeptide encoded by the nucleic acid sequence of SEQ ID No. 1 can deviate from the polypeptide of SEQ ID NO:2. For example, a recoding would be given when expressing polynucleotide set out in SEQ ID NO:1 in the yeast Candida, in which the code CUG, which normally codes for Leucine is read as Serine.

In a further aspect of the invention, the ITIH5 polypeptide is a biologically active fragment, analog, variant, derivative or elongation of the polypeptide encoded by the polynucleotide set out in SEQ ID NO:1.

In another aspect of the invention, the ITIH5 polypeptide is a polypeptide encoded by a polynucleotide that hybridizes to the polynucleotide set out in SEQ ID NO:9 under moderately stringent hybridization conditions. The SEQ ID NO:9 represents the reverse complementary nucleic acid sequence of the polynucleotide set out in SEQ ID NO:1. Hence, any polynucleotide hybridizing to the polynucleotide set out in SEQ ID NO:9 has the 5′ to 3′ orientation representing the coding sequence of the respective ITIH5 polypeptide.

The isolated or purified ITIH5 polypeptide of the present invention has the biological activity of a negative regulator of adipogenesis. This biological activity is defined by at least one of the following two biological effects:

The first biological effect is the inhibition of the insulin-induced adipogenic differentiation of human adipose stem cells (ASCs) into mature adipocytes by addition of the ITIH5 polypeptide (or a fragment, analog, variant, derivative or elongation thereof) to in vitro-cultured human ASCs. The respective bio-assay is described in Example 6 below.

The second biological effect is the inhibition of proliferation of human adipose stem cells (ASCs) by addition of the ITIH5 polypeptide (or a fragment, analog, variant, derivative or elongation thereof) to in vitro-cultured human ASCs. The respective bio-assay is described in Example 5 below.

In a preferred embodiment of the invention, the ITIH5 polypeptide of the present invention exhibits both biological effects, namely the inhibition of the insulin-induced adipogenic differentiation of human adipose stem cells (ASCs) into mature adipocytes and inhibition of proliferation of human ASCs.

According to the present invention, the isolated or purified ITIH5 polypeptide is used for reducing weight or for reducing weight gain in the subject to be treated. This phenotypic result can be achieved by multiple cellular/physiological mechanisms such as:
a) reducing the number of adipose stem/stroma cells, preadipocytes, adipocytes. This effect can be deduced from the antiproliferative effect of ITIH5 as shown in Example 5 below.
b) reducing differentiation, dysregulated differentiation, and maturation of adipose stem/stroma cells and preadipocytes into mature adipocytes. This effect can be deduced from the inhibition of the insulin-induced adipogenic differentiation of human adipose stem cells (ASCs) into mature adipocytes by ITIH5 as shown in Example 6 below.
c) reducing the development, enlargement, and fusion of fat vacuoles and supersized lipid droplets. Adipose connective tissue cells are specialized for fat storage and do not form ground substance or fibers. Anatomically, adipose tissue appears somewhat like a fish net with white spaces connected together in a network. The cytoplasm and nucleus have been pushed to one side by a single, large, fat-filled vacuole that occupies the center of the cell, whereby the dimension of the fat vacuole is indicative of the obesity status.
d) reducing the amount of adipose tissue. A reduced ASC proliferation and differentiation should accordingly reduce the amount of adipose tissue.
e) inhibiting dysfunction of adipose tissue. Insofar the dysfunction of adipose tissue is often given by abnormal proliferation and differentiation of ASCs, this should be cured by the ITIH5 polypeptide of the present invention.
f) reducing the risk for impaired insulin sensitivity and systemic metabolic deterioration in obese state, e.g., type 2 diabetes, non-alcoholic fatty liver disease, dyslipidemia, hypertension or lipotoxicity. By tackling the obesity as primary source of several metabolic disorders, the ITIH5 polypeptide of the present invention has also the potential to prevent or treat the aforementioned metabolic disorders and syndromes.

The term subject as used herein includes all members of the animal kingdom. In a preferred embodiment of the present invention, the subject to the treated by the isolated or purified ITIH5 polypeptide is a mammal and preferable a human subject.

In a preferred embodiment, the subject to be treated by ITIH5 polypeptide is an obese or overweight subject. Overweight and obesity are characterized by abnormal or excessive fat accumulation that presents a risk to health. A body mass index (BMI) over 25 is considered overweight, and over 30 is obese.

The ITIH5 polypeptide according to the present invention is used for the treatment of obesity. The term “obesity” as used herein encompasses human subjects with a BMI over 30 and also overweight subjects with a BMI over 25. Furthermore, it encompasses the obesity related attendant symptoms and obesity-induced metabolic disease, and therefore includes the one of the following: inflammation, cardiovascular disease, hypertension, dyslipidemia, heart failure, atherosclerosis, coronary artery disease, stroke, diabetes mellitus type 2, prediabetes, insulin resistance, insulin resistance syndrome (also known as Syndrome X), gall bladder disease, osteoarthritis, sleep apnoea, liver steatosis, obesity-associated liver steatosis, liver inflammation, liver fibrosis, diabetes, obesity-associated fat accretion, obesity-related cancer, infections, varicose veins, acanthosis nigricans, eczema, exercise intolerance, hypertension hypercholesterolemia, cholelithiasis, and decreased life expectancy.

In one preferred embodiment, the ITIH5 polypeptide according to the present invention is used for the treatment of inflammation as an obesity-related symptom. As shown in Example 7 and FIG. 7, the inventors found that ITIH5 inhibited the release of inflammatory cytokines from ASCs exposed to an artificial inflammatory environment, mimicked by the presence of TNF-α and IL-1β. Thus, in the setting of adipose tissue development, ITIH5 secretion by mature adipocytes could exert regulating effects on metabolic dysfunction by controlling inflammatory responses of ASCs in a paracrine way.

In one embodiment, the subject to be treated with the isolated or purified ITIH5 polypeptide of the invention does not has diabetes.

Additional embodiments of the present disclosure encompass compositions comprising the isolated ITIH5 polypeptides. The ITIH5 polypeptide may be formulated into pharmaceutical compositions for administration to subjects in a biologically compatible form suitable for administration. By biologically compatible form suitable for administration is meant a form of the substance to be administered in which any toxic effects are outweighed by the therapeutic effects. The substances may be administered to living organisms, including humans and animals, in a therapeutically effective amount. Administration of an effective amount of the pharmaceutical compositions of the present invention is defined as an amount effective, at dosages and for periods of time necessary to achieve the desired result. For example, a therapeutically active amount of a substance may vary according to factors such as the disease state, age, sex and weight of the individual, and the ability of peptide to elicit a desired response in the individual. Dosage regime may be adjusted to provide the optimum therapeutic response. For example, several divided doses may be administered daily or the dose may be proportionally reduced as indicated by the exigencies of the therapeutic situation. The active substance may be administered in a convenient manner such as by topical or transdermal application, injection (subcutaneous, intravenous, etc.), oral administration, inhalation, or rectal administration. Depending on the route of administration, the active substance may be coated in a material to protect the compound from the action of enzymes, acids and other natural conditions which may inactivate the compound.

Compositions for injection include, albeit not exclusively, the ITIH5 polypeptide in association with one or more pharmaceutically acceptable vehicles or diluents, and contained in buffered solutions with a suitable pH and iso-osmotic with the physiological fluids. Any pharmaceutically suitable diluent can be used in the composition for injections: distilled water, physiological or a salt solution, and/or a buffer solution. The composition for injections may be prepared by conventional volume-weight procedures. A certain amount of the peptide is diluted to the necessary volume with a diluent or solvent. The solution is then filtered through sterilized filters, bottled or ampouled. The resultant solution is a stable transparent liquid and does not contain any chemical or other impurities.

The isolated or purified ITIH5 polypeptide of the present invention can be applied by any route known by the skilled person for application of peptides. In a preferred embodiment, the route of administration of the ITIH5 polypeptide is intravenous, intraperitoneal, intramuscular, subcutaneous, topical, nasal or pulmonary inhalation.

Solid form preparations for oral administration can be made in the form of tablets, powders, or capsules. It may contain a medium for the active substance and other additives, including dyes, aromas, etc.

In a further aspect, the invention relates to a pharmaceutical composition comprising the isolated or purified ITIH5 polypeptide of the invention together with at least one pharmaceutically acceptable excipient. The term “pharmaceutical composition” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

In a further aspect, the invention relates to a method for identifying epitopes of the ITIH5 protein that reduce weight or reduce weight gain of a subject by modulation of the ITIH5-adipogenic pathway, comprising the following steps:
a. Synthesizing small peptides with a length of 20 to 40 amino acids covering the whole ITIH5 protein or an adipogenesis-active fragment thereof in an overlapping manner;
b. contacting individual small peptides separately to in vitro cultured adipose stem cells and further incubating the ASCs a preselected time of cell culture under conditions that allow ASC differentiation;
c. determining the ASC differentiation after a preselected time of cell culture by staining of the cells with a fat-soluble dye that stains neutral triglycerides and lipids such as OilRed®;
d. selection of small peptides inhibiting ASC differentiation as epitopes of ITIH5 that are capable of reducing weight or reduce weight gain of a subject by modulation of the ITIH5-adipogenic pathway.

In another aspect the invention relates to a method for identifying epitopes of the ITIH5 protein that reduce weight or reduce weight gain of a subject by modulation of the ITIH5-adipogenic pathway, comprising:
a. Synthesizing small peptides with a length of 20 to 40 amino acids covering the whole ITIH5 protein or a proliferation-active fragment thereof in an overlapping manner;
b. contacting individual small peptides separately to in vitro cultured adipose stem cells and further incubating the ASCs a preselected time of cell culture under conditions that allow ASC proliferation;
c. determining the ASC proliferation by measuring the cell numbers such as Crystal Violet staining using a detection platform including fluorescence microscopy, flow cytometry, and microplate readers;
d. selection of small peptides inhibiting ASC proliferation as epitopes of ITIH5 that are capable of reducing weight or reduce weight gain of a subject by modulation of the ITIH5-proliferative pathway.

In a further aspect, the invention relates to a method for identifying synthetic peptides of the ITIH5 protein that reduce weight or reduce weight gain of a subject by modulation of the ITIH5-adipogenic pathway, by selecting a bioactive ITIH5 epitope as identified by one of the two methods disclosed above.

In another aspect, the invention relates to a method for identifying mimetic small molecules that reduce weight or reduce weight gain of a subject by modulation of the ITIH5-adipogenic pathway, comprising:
a. providing a library of small organic compounds,
b. providing adipocyte precursor cells (APCs) and transfecting them with an expression vector leading to ITIH5 overexpression,
c. selecting single clones from the cells transfected in step b. showing ITIH5 overexpression,
d. providing the adipocyte precursor cells (APCs) as chosen in step b. and transfecting with the expression vector of step. b. not containing the ITIH5 expression construct (control vector) leading to control APCs,
e. selecting single clones from the cells transfected in step d., not exhibiting ITIH5 overexpression (control APCs),
f. in vitro-cultivating at least one clone of the ITIH5-overexpressing APCs and at least one clone of the control APCs and contacting cells from each clone individually with the same small organic compounds of the library provided in step a.,
g. analysing the proliferation or the fat cell differentiation of the clones subjected to the small organic compounds of step f, whereas a ITIH mimicking compound leads to a reduction in APC proliferation or fat cell differentiation in the control APC but not in the ITIH5-overexpressing APCs.

In the context of the present invention, a small organic compound is an organic compound with a molecular weight of less than 900 daltons and preferably a molecular weight of less than 500 daltons. In a preferred embodiment the small organic compound obeys the Lipinski's rule of five. This mean that in order to constitute an orally active drug, the compound has no more than one violation of the following four criteria:
No more than 5 hydrogen bond donors (the total number of nitrogen-hydrogen and oxygen-hydrogen bonds),
No more than 10 hydrogen bond acceptors (all nitrogen or oxygen atoms),
A molecular mass less than 500 daltons,
A calculated octanol-water partition coefficient (C log P) that does not exceed 5.

The adipocyte precursor cells (APCs) of step b. are cells that could lead to in vitro-differentiation into white adipocytes. Several APCs are known to the skilled person such as human adipose stem cells (ASC) or mouse 3T3-L1 cells. Mouse 3T3-L1 cells are widely used as an in vitro model of white adipocyte differentiation.

The transfection in step b. can be a transient or stable transfection. Transiently transfected cells express the foreign ITIH5 gene construct but do not integrate it into their genome. Thus, the new ITIH5 gene construct will not be replicated. These cells express the transiently transfected ITIH5 gene construct for a finite period of time, usually several days, after which the foreign ITIH5 gene construct is lost through cell division or other factors. The hallmark of stably transfected cells is that the foreign ITIH5 gene construct becomes part of the genome and is therefore replicated. Descendants of these transfected cells, therefore, will also express the new ITIH5 gene construct, resulting in a stably transfected cell line.

The expression vector of step b. leading to ITIH5 overexpression is a mammalian protein expression vector containing the ITIH5 coding sequence under the control of a suitable promotor allowing ITIH5 overexpression (such as for example the CMV enhancer-promotor).

The expression vector used in step b. can be a plasmid (e.g. pCDNA3.1-ITIH5) or viral (e.g. lentiviral system) expression vector.

The ITIH5 overexpression of the transfected cells as described in step c. can be validated by standard techniques such as quantitative PCR or Western blot.

The control vector of step d. does not contain the ITIH5 expression construct. In the easiest version it the same expression vector which is used for the ITIH-5 overexpression with the difference, that no additional gene is cloned into it. For example, when using the pCDNA3.1-ITIH5 vector, the original pCDNA3.1 vector could be used as control vector. As an alternative, the control vector could be equipped with a marker gene.

For the compound screening as described in steps f. and g. at least one, preferably two or more two high expresser clones (ITIH5 positive clones) are compared to at least one, preferably two or more control clones, being transfected only with the vector system (ITIH5 negative clones).

As described in step g. the following compounds are considered as functional ITIH5 mimetic candidates: A small organic compound under consideration leads to a reduction in APC proliferation and fat cell differentiation (e.g. determined by measuring lipid content using Oil Red O, or Nile Red staining) in ITIH5 negative clones (where it mimics ITIH5 action) but not in ITIH5 positive clones (where no effect is observed because ITIH5 is already present).

The APC proliferation as measured in step g. can be performed by any method known by the skilled person in the context of in vitro-cultivated mammalian cells. Non-restrictive examples of such methods are cell counting assays or Crystal violet staining.

The fat cell differentiation and proliferation as measured in step g. can be performed by any method known by the skilled person in the context of in vitro-cultivated APCs. Non-restrictive examples of such methods measure the lipid content of the cells by staining with appropriate dyes such as Oil Red O or Nile Red.

Candidate ITIH5 mimetics as identified by the screening method of the invention can be validated further for activity and specificity by measuring its action in comparison to recombinant ITIH5 protein applied to APCs. Furthermore, gene profiling experiments comparing ITIH5 positive and negative APCs can be performed to determine target molecules regulated by ITIH5 in APCs. Those candidate ITIH5 mimetics (small molecule compounds) that regulate the same target molecules as ITIH5 protein may be most suitable for therapeutic intervention of obesity.

In an alternative embodiment, the small molecule screening approach as described above may be performed using APCs being kept constantly under optimal concentration of recombinant ITIH5 versus APCs under diluent control. The methods for selection suitable hits for ITIH5 mimetics are the same as described above. The present disclosure is also directed to vectors comprising a nucleotide molecule of the present disclosure. The term “vector” includes a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

The present disclosure is also directed to a host cell with a vector comprising the recombinant ITIH5 polypeptide according to the present disclosure. The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells include progeny of a single host cell, and the progeny may not necessarily be completely identical (in morphology or in total DNA complement) to the original parent cell due to natural, accidental, or deliberate mutation and/or change. A host cell includes a cell transfected or infected in vivo or in vitro with a recombinant vector or a polynucleotide of the present disclosure. A host cell, which comprises a recombinant vector of the present disclosure may also be referred to as a “recombinant host cell”.

The term “host cell(s)” refers to cell(s), which may be used in a process for purifying a recombinant ITIH5 polypeptide in accordance with the present disclosure. Such host cells carry the protein of interest (POI). A host cell may also be referred to as a protein-expressing cell. A host cell, according to the present invention, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsiella sp., Lactobacillus sp., Candida albicans or Bacillus subtilis. Typical yeast host cells are selected from the group consisting of Saccharomyces cerevisiae, Hansenula polymorpha and Pichia pastoris.

To express a ITIH5 polypeptide according to the present disclosure, a DNA encoding the ITIH5 polypeptide or parts thereof, may be inserted into an expression vector such that the ITIH5 gene is operably linked to transcriptional and translational control sequences. In this context, the term “operably linked” means that a protein gene is ligated into a vector such that transcriptional and translational control sequences within the vector serve their intended function of regulating the transcription and translation of the protein gene. The expression vector and expression control sequences are chosen to be compatible with the expression host cell used. The isolated protein domain sequences are typically inserted into the same expression vector. The protein genes are inserted into the expression vector by standard methods. Additionally, the recombinant expression vector can encode a signal peptide that facilitates co-translational translocation of the nascent polypeptide chain into the endoplasmic reticulum (ER). The polypeptide or peptide may be secreted from a host cell or may be retained within the host cell. Intracellular retention or targeting can be achieved by the use of an appropriate targeting peptide such as C-terminal KDEL-tag for ER retrieval.

In general, those skilled in the art are well able to construct vectors and design protocols for recombinant gene expression. For further details see, for example, Molecular Cloning: A Laboratory Manual: 2nd edition, Green M R & Sambrook J, 2001, Cold Spring Harbor Laboratory Press and Current Protocols in Molecular Biology, Second Edition, Ausubel et al. eds., John Wiley & Sons, 1992, which are incorporated herein by reference.

Further aspects of the disclosure relate to a method of expressing in a host cell a recombinant ITIH5 polypeptide thereof as described herein from a nucleic acid molecule described herein; a host cell capable of expressing a ITIH5 polypeptide as described herein in appropriate culture conditions for producing said polypeptide or peptide; a method of producing a ITIH5 polypeptide comprising culturing such a host cell under appropriate conditions, which method may further comprise isolating said ITIH5 polypeptide from the cell culture, and which method may further comprise admixing the isolated ITIH5 polypeptide with a suitable further component (which may, for example, be another protein or an excipient or carrier).

The produced ITIH5 polypeptides according to the present disclosure may be recovered, further purified, isolated, processed and/or modified by methods known in the art. For example, the polypeptide may be recovered from the nutrient medium by conventional procedures including, but not limited to, centrifugation, filtration, ultra-filtration, extraction or precipitation.

Further processing steps such as purification steps may be performed by a variety of procedures known in the art including, but not limited to, chromatography (e.g. ion exchange, affinity, hydrophobic, chromatofocusing, and size exclusion), electrophoretic procedures (e.g., preparative isoelectric focusing), differential solubility (e.g. ammonium sulfate precipitation) or extraction.

Furthermore, the isolated and purified ITIH5 polypeptide of interest may be further processed, such as e.g. formulated into a composition, e.g. a pharmaceutical composition.

As mentioned above, a nucleic acid molecule of the present disclosure may also be chemically synthesized using standard techniques. Various methods of chemically synthesizing polydeoxynucleotides are known, including solid-phase synthesis which, like peptide synthesis, has been fully automated in commercially available DNA synthesizers.

It should also be expressly noted that, in the context of the present patent application, indefinite articles and numerical indications such as “a”, “an”, “one”, “two”, etc. are generally to be understood as “at least”-indications, i.e. as “at least one . . . ”, “at least two . . . ”, etc., unless it expressly follows from the respective context or it is obvious or technically imperative to a person skilled in the art that only “exactly one . . . ”, “exactly two . . . ”, etc. can be meant there.

Further advantages, features and details of the invention will be apparent from the following description, in which embodiments of the invention are described in detail with reference to the figures.

The foregoing explanation of the embodiments describes the present invention exclusively in the context of examples. Of course, individual features of the embodiments can be freely combined with each other, provided that this is technically reasonable, without leaving the scope of the present invention.

### Definitions

Obesity: The term “obesity” as used herein encompasses human subjects with a BMI over 30 and also overweight subjects with a BMI over 25. Furthermore, it encompasses the obesity related attendant symptoms and obesity-induced metabolic diseases as described herein.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Essentially: The term “essentially” is to be understood that methods or compositions include only the specified steps or materials and those that do not materially affect the basic and novel characteristics of those methods and compositions.

Approximately: The term “approximately” as used herein may be applied to modify any quantitative comparison, value, measurement, or other representation that could permissibly vary without resulting in a change in the basic function to which it is related.

Or/and/or: The use of the term “or” in the claims is used to mean “and/or” unless explicitly indicated to refer to alternatives only or the alternatives are mutually exclusive, although the disclosure supports a definition that refers to only alternatives and “and/or.”

The term “about” means, in general, within a standard deviation of the stated value as determined using a standard analytical technique for measuring the stated value. The terms can also be used by referring to plus or minus 5% of the stated value.

Protein/polypeptide: The terms “protein,” “peptide,” and “polypeptide,” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

In an advantageous embodiment, the ITIH5 polypeptide of the present disclosure is an isolated polypeptide. The term “isolated” when used in relation to a protein (e. g. an protein domain), refers to a protein that is identified and separated from at least one contaminant (e.g. proteins, cells or other cell constituents) with which it is ordinarily associated in its natural source. Accordingly, an isolated protein is present in a form or setting that is different from that in which it is found in nature. In contrast, non-isolated proteins are found in the state they exist in nature.

Variant: The term “variant” as used for the ITIH5 polypeptide refers to variants of genes or transcripts which originate from the same genetic ITIH5 locus as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Said natural variants occur in nature and are not artificially generated. In contrast, when the term “variant” is used without the word “natural” being associated to it, the variants may comprise artificially generated variants such as mutations. In the case of human ITIH5 some variants are the isoform 2 of SEQ ID NO:6, the isoform 3 of SEQ ID NO:7 and the isoform of SEQ ID NO:8.

The term “variant” means that the amino acid sequence has been modified but retains the same functional characteristics, in particular the growth inhibitory effect on human cancer cells. A variant has a sequence identity of at least 95%, 97% or 99% to the parent amino acid sequence.

The term “analog” includes any peptide having an amino acid residue sequence substantially identical to the sequence of the peptides shown herein in which one or more residues have been conservatively substituted with a functionally similar residue and which displays the ability to mimic a peptide according to the present disclosure. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

The term “derivative” refers further to a polypeptide or peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imidazolebenzyl-histidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3methylhistidine may be substituted for histidine; homoserine may be substituted for serine; and ornithine may be substituted for lysine. Polypeptides and/or peptides comprising the vWFA and/or VIT domain of ITIH5 or peptides derived from these domains also include any polypeptide or peptide having one or more additions and/or deletions or residues relative to the sequence of a polypeptide or peptide whose sequence is shown herein, so long as the requisite activity is maintained or increased.

The term “elongation” refers to any subject peptide having additional amino acid residues added to either end of the peptide, preferably from 1 to 10 amino acid residues, added to either the amino-terminal and/or carboxy-terminal end of a peptide of the present disclosure.

The ITIH5 polypeptides according to the present disclosure are characterized by specific amino acids and is encoded by specific nucleic acid sequences. It will be understood that such sequences include analogues and variants produced by recombinant or synthetic methods wherein such polypeptide sequences have been modified by substitution, insertion, addition or deletion of one or more amino acid residues in the recombinant polypeptide and still shows an antiproliferative or differentiation-inhibiting effect on human adipose stem cells in any of the biological assays described herein. Substitutions are preferably “conservative”. Substitutions are preferably silent substitutions in the codon usage, which will not lead to any change in the amino acid sequence but may be introduced to enhance the expression of the protein.

Nucleic acid: The term “nucleic acid” includes any nucleotides, analogues thereof, and polymers thereof. The terms “polynucleotide” or “nucleotide sequence” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogues of either RNA or DNA made from nucleotide analogues and modified polynucleotides such as, though not limited to, methylated, protected and/or capped nucleotides or polynucleotides. The terms encompass poly- or oligo-ribonucleotides (RNA) and poly- or oligo deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges. The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxyribose moieties, nucleic acids containing both ribose and deoxyribose moieties, nucleic acids containing ribose and modified ribose moieties. In some embodiments, the prefix poly-refers to a nucleic acid containing 2 to about 10,000, 2 to about 50,000, or 2 to about 100,000 nucleotide monomer units. In some embodiments, the “oligonucleotide” refers to a nucleic acid containing 2 to about 200 nucleotide monomer units.

Gene: As used herein, the term “gene” means a segment of DNA that contains all the information for the regulated biosynthesis of an RNA product, including promoters, exons, introns, and other untranslated regions that control expression. Also, shRNAs are included by this definition.

Hybridize: The terms “hybridizing” or “hybridizes” as used herein is to be understood as two nucleic acid strands (e.g., an oligonucleotide and a target nucleic acid) forming hydrogen bonds between base pairs on opposite strands thereby forming a duplex.

Polynucleotides encoding fragments, variants and analogs may be readily generated by a worker of skill to encode biologically active fragments, variants, or analogs of the naturally-occurring molecule that possess the same or similar biological activity to the naturally-occurring molecule. These polynucleotides can be prepared using PCR techniques, digestion/ligation of DNA encoding molecule, and the like. Thus, one of skill in the art will be able to generate single base changes in the DNA strand to result in an altered codon and a missense mutation, using any method known in the art, including, but not limited to site-specific mutagenesis. As used herein, the phrase “moderately stringent hybridization conditions” means, for example, hybridization at 42° C. in 50% formamide and washing at 60° C. in 0.1×SSC, 0.1% SDS. It is understood by those of skill in the art that variation in these conditions occurs based on the length and GC nucleotide base content of the sequences to be hybridized. Formulas standard in the art are appropriate for determining exact hybridization conditions. Green and Sambrook, Molecular Cloning: A Laboratory Manual, 2012, 4th ed., Cold Spring Harbor Laboratory Press, Cold Spring Harbor, N.Y., Chapter 9.47-9.51).

Mutation: The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. 2012).

“Percent sequence identity”, with respect to two amino acid or polynucleotide sequences, refers to the percentage of residues that are identical in the two sequences when the sequences are optimally aligned. Thus, 80% amino acid sequence identity means that 80% of the amino acids in two optimally aligned polypeptide sequences are identical. Percent identity can be determined, for example, by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN14, National Biomedical Research Foundation, Washington, DC, which adapts the local homology algorithm of Smith and Waterman for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters 5 recommended by the manufacturer and described in the Wisconsin Sequence Analysis Package referred to above. An example of an algorithm that is suitable for determining sequence similarity is the BLAST algorithm. Software for performing BLAST analyses is publicly available through the National Center for Biotechnology Information (http://www.ncbi.nlm.nih.gov/). Likewise, computer programs for determining percent homology are also readily available.

Recombinant: As used herein, the term “recombinant” is intended to refer to nucleic acids or polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Wildtype: As used herein “wildtype” refers to the naturally occurring sequence of a nucleic acid at a genetic locus in the genome of an organism, and sequences transcribed or translated from such a nucleic acid. Thus, the term “wildtype” also may refer to the amino acid sequence encoded by the nucleic acid. As a genetic locus may have more than one sequence or alleles in a population of individuals, the term “wildtype” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology.

Subject: As used herein, the terms “subject”, “test subject” or “patient” refer to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically engineered animal, and/or a clone.

Treatment/treating: “Treatment” or “treating” includes (1) inhibiting a disease, disorder or condition in a subject or patient experiencing or displaying the pathology or symptomatology of the disease (e.g., arresting further development of the pathology and/or symptomatology), (2) ameliorating a disease, disorder or condition in a subject or patient that is experiencing or displaying the pathology or symptomatology of the disease (e.g., reversing the pathology and/or symptomatology), and/or (3) effecting any measurable decrease in a disease, disorder or condition in a subject or patient that is experiencing or displaying the pathology or symptomatology of the disease.

Prophylactically treating: “Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

Local administration/local delivery: As used herein, the term “local administration” or “local delivery”, in reference to delivery of a recombinant expression system, an expression vector, a viral particle or a pharmaceutical composition described herein, refers to delivery that does not rely upon transport of said products to its intended target tissue or site via the vascular system. Said products described herein may be delivered directly to its intended target tissue or site, or in the vicinity thereof, e.g., in close proximity to the intended target tissue or site. For example, said products may be delivered by injection or implantation of the composition or compound or by injection or implantation of a device containing the composition or compound. Following local administration in the vicinity of a target tissue or site, said products described herein, or one or more components thereof, may diffuse to the intended target tissue or site. It will be understood that once having been locally delivered a fraction of said products described herein (typically only a minor fraction of the administered dose) may enter the vascular system and be transported to another location, including back to its intended target tissue or site. As used herein, the term “local administration” or “local delivery”, in reference to delivery of a viral particle or a pharmaceutical composition described herein, refers to delivery that can rely upon transport of the viral vector or the pharmaceutical composition to its intended target tissue or site via the vascular system.

Effective/therapeutically effective amount: As used herein, the term “effective” or “therapeutically effective amount” means at least the minimum amount of a compound (e.g., a therapeutic agent, expression vector, viral particle, pharmaceutical composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount. A therapeutically effective amount is also one in which any toxic or detrimental effects of the treatment are outweighed by the therapeutically beneficial effects. In the case of metabolic disorder or obesity, an effective amount of a compound may have the effect in reducing the weight or weight gain, and/or relieving to some extent one or more of the obesity attendant symptoms or the metabolic disease induced by obesity.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWINGS

FIG. 1 illustrates in a) the body weight, in b) the absolute fat weight (from explanted fat tissue of inguinal region and gonads), and in c) the relative fat weight as amount of fat pad to total body weight, each shown for wildtype mice (WT) as compared to ITIH5 knock-out mice (ITIH5^{-/-}). The data are present as median value, whereby the box contains 50% of the measured values and is delimited by the upper and lower quartile (25% or 75%, respectively are lower or equal to this value). The maximum and minimum values are displayed with vertical lines (“whiskers”). The data were statistically analysed using the Mann-Whitney U test. The genetic elimination of ITIH5 gene and thus ITIH5 protein significantly increases the relative fat weight (*p<0.05).

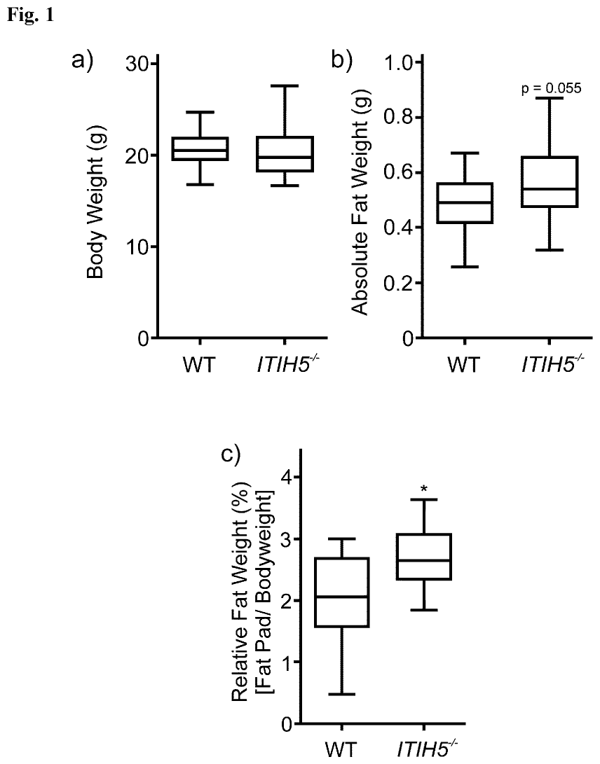

FIG. 2 illustrates the number of in vitro cultivated ASCs from wildtype mice (WT) or ITIH5 knock-out mice (ITIH5^{-/-}) after a) 7 days, or b) 14 days, as measured with Crystal violet staining (n=3 mice per group; N=51 measurements). The data are present as median value, whereby the box contains 50% of the measured values and is delimited by the upper and lower quartile (25% or 75%, respectively are lower or equal to this value). The maximum and minimum values are displayed with vertical lines (“whiskers”). The data were statistically analysed using the Mann-Whitney U test. The genetic elimination of ITIH5 gene and thus ITIH5 protein significantly increases the proliferation of the ASCs (***p<0.001).

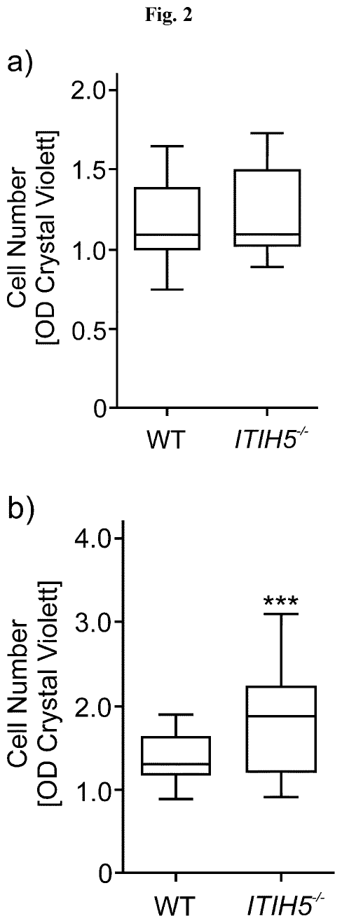

FIG. 3 illustrates the adipogenic differentiation of in vitro cultivated ASCs from wildtype mice (WT) or ITIH5 knock-out mice (ITIH5^{-/-}) after 14 days of cultivation as measured with Oil Red staining. WT: n=1 and N=51; ITIH5^{-/-}: n=2, N=37. The data are presented as median value, whereby the box contains 50% of the measured values and is delimited by the upper and lower quartile (25% or 75%, respectively are lower or equal to this value). The maximum and minimum values are displayed with vertical lines (“whiskers”). The data were statistically analysed using the Mann-Whitney U test. The genetic elimination of ITIH5 gene and thus ITIH5 protein significantly increases the adipogenic differentiation of the ASCs (***p<0.001).

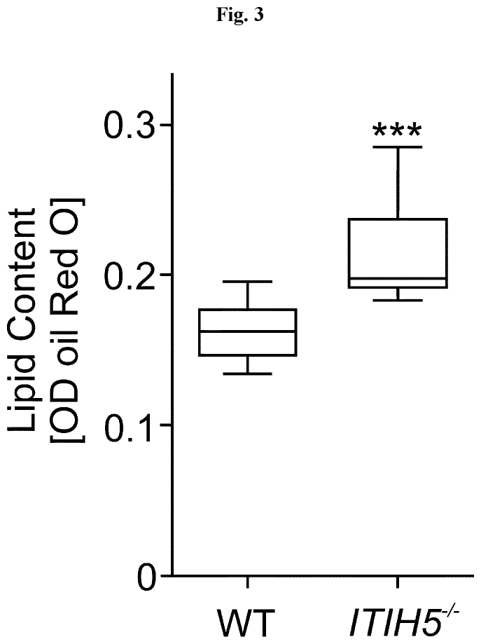

FIG. 4 illustrates the concentration of ITIH5 protein (in pg) per mg of total protein in human adipose tissue as a function of the body mass index (BMI) of the respective human tissue donor (n=13 donors). The data were statistically analysed using the Pearson test. The correlation between ITIH5 protein concentration and BMI is significant (**p<0.006).

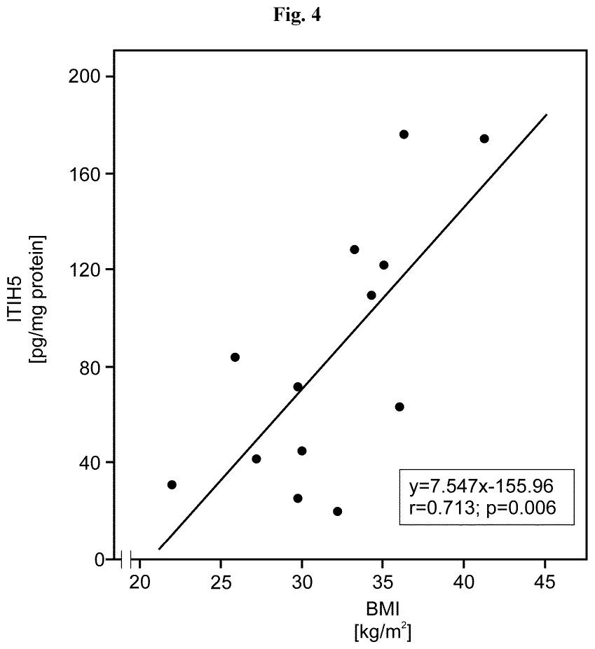

FIG. 5 illustrates the number of in vitro cultivated human ASCs after a) 7 days, or b) 14 days of cultivation, without stimulation (ctrl) or after addition of recombinant human ITIH5 in a concentration of 0.01 or 0.1 μg protein/ml cell culture medium (n=4 donors, N=51 measurements). The data are present as median value, whereby the box contains 50% of the measured values and is delimited by the upper and lower quartile (25% or 75%, respectively are lower or equal to this value). The maximum and minimum values are displayed with vertical lines (“whiskers”). The data were statistically analysed using the Mann-Whitney U test versus control. The ITIH5 protein significantly decreases the proliferation of human ASCs (*p<0.05; ***p<0.001).

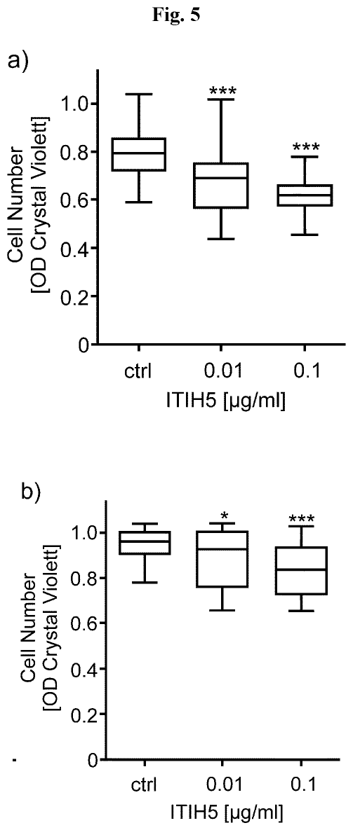

FIG. 6 illustrates the adipogenic differentiation of in vitro cultivated human ASCs after 14 days of cultivation, without stimulation (ctrl) or after addition of recombinant human ITIH5 in a concentration of 0.01 or 0.1 μg protein/ml cell culture medium (n=4 donors, N=64 measurements). The respective measurements were normalized with regard to the respective cell numbers by division to their OD of the crystal violet staining after 14 days. The data are presented as median value, whereby the box contains 50% of the measured values and is delimited by the upper and lower quartile (25% or 75%, respectively are lower or equal to this value). The maximum and minimum values are displayed with vertical lines (“whiskers”). The data were statistically analysed using the Mann-Whitney U test versus control. The ITIH5 protein significantly reduces the adipogenic differentiation of human ASCs (***p<0.001).

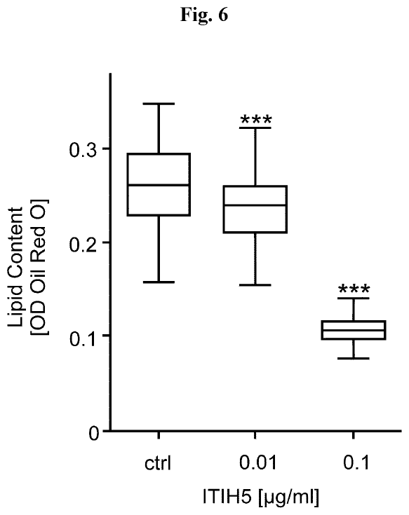

FIG. 7. Effect of recombinant ITIH5 protein stimulation on cytokine release when undifferentiated ASCs were exposed to inflammatory mediators. Cells were either untreated (−), or stimulated with recombinant ITIH5 protein (at 0.01 or 0.1 μg/ml, as indicated); ASCs were treated with 1 ng/ml TNF-α or IL-1β, or co-exposed to both (+). After 48 h, media were collected to determine cytokine secretion by sandwich ELISA: IL-6 (a-d), MCP-1 (e-h). Data were calculated per cell number (divided by OD crystal violet—CV), and are presented as mean values (+SEM). The number of experiments and donors were as follows: n≥18 (4 donors). Statistical analyses were performed by pairwise comparison vs. cells without ITIH5 protein exposure (−) using the Student's t-test; *p≤0.05, **p≤0.01, and ***p≤0.001.

### EXAMPLE 1: EFFECT OF ITIH5 GENE KNOCKOUT ON ADIPOSE TISSUE DEVELOPMENT IN MICE

The objective of this experiment was to examine the body weight, and the absolute and relative fat weight of wildtype and ITIH5-knockout mice.

Wildtype mice (BALB/c) were purchased from Charles River Laboratories International (Wilmington, MA). ITIH5^{-/-} mice (same strain) were generated by TaconicArtemis (Cologne, Germany). All mice were held in the facilities of the Institute of Laboratory Animal Science of the University Hospital, RWTH Aachen. Mice were sacrificed for experimental investigation at the age of 8-9 weeks with animals (n=31) of both sexes. After cervical dislocation in deep anesthesia (5% isoflurane), skin of mice was disinfected with 70% ethanol. Adipose samples were acquired from the s.c. inguinal and gonadal fat depots. The animal experiments were performed according to EU Directive 2010/63/EU for animal experiments, they followed the guidelines of the animal welfare laws and were approved by the Animal Care and Use Committee of the state of North Rhine-Westphalia, Germany. Bodyweight and fat weight were determined using a laboratory balance (Kern, Lörrach, Germany).

The genetic elimination of the ITIH5 gene and thus ITIH5 protein significantly increased the relative fat weight in ITIH5 knockout mice.

### EXAMPLE 2: ANALYSIS OF ASC PROLIFERATION IN ITIH5^{-/-} (KNOCKOUT) VS. WILD TYPE MICE

The objective of this experiment was to examine the proliferative activity of adipose stem cells from wildtype and homozygous (ITIH5^{-/-}) knockout mice.

Adipose samples from mice were acquired from the gonadal and inguinal fat depots, which were completely resected before enzymatic digestion. Adipose tissue was minced with surgical scissors, washed with PBS, and digested in 0.2% collagenase for 45 min at 37° C. The stromal cell fraction was separated by centrifugation at 400×g, and filtered through a 250 nm nylon mesh (neoLab, Heidelberg, Germany). After centrifugation, cells were cultured in proliferation medium (DMEM supplemented with 10% FBS, 0.1% bFGF). Experiments were conducted with cells from P2-P4 seeded at a density of 20.000 per cm².

Proliferation was assessed after 7 and 14 days of standard culture by measuring cell numbers using crystal violet staining. Cells were washed with PBS and fixed in isopropyl alcohol for 10 min at RT, followed by washing with PBS containing 0.05% Tween20. Then, cells were stained with 0.1% crystal violet for 20 min. After removal of the crystal violet solution, plates were washed with aqua_{bidest}. Adsorbed dye was washed out in 33% acetic acid during 15 min incubation with gentle agitation. 70 μl per sample were transferred in triplets to an optical plate and absorbance was quantified in a microplate reader (BMG Labtech, Ortenberg, Germany) at 620 nm.

The genetic elimination of the ITIH5 gene and thus ITIH5 protein significantly increased the proliferation of the ASCs.

### EXAMPLE 3: ANALYSIS OF ADIPOGENIC DIFFERENTIATION IN ITIH5^{-/-} VS. WILD TYPE MICE

The objective of this experiment was to examine the capability of adipogenic differentiation of adipose stem cells from wildtype and ITIH5-knockout mice.

For adipogenic differentiation, cells were exposed to differentiation medium: high glucose medium supplemented with 2% FBS, 10 μM insulin, 0.28 μM rosiglitazone. The media were replaced every 2 to 3 days for 14 days. Adipogenic differentiation was assessed by OilRed O staining. ASCs were PFA fixed, and stained with 0.2% OilRed O solution. Adsorbed dye was washed out with isopropanol. Absorbance was measured in triplets at 540 nm.

The genetic elimination of the ITIH5 gene and thus ITIH5 protein significantly increases the adipogenic differentiation of the ASCs.

### EXAMPLE 4: ANALYSIS OF ITIH5 PROTEIN CONTENT IN HUMAN ADIPOSE TISSUE OF OBESE PATIENTS

The objective of this experiment was to examine the content of ITIH5 protein in human adipose tissue in relation to the body mass index (BMI) of obese human subjects.

Human adipose tissue samples were cut en bloc during abdominoplasty and either frozen for later determination of protein levels, or processed for isolation of adipose stem cells. The healthy donors had been informed about the utilization of their tissue and had given informed consent. The study protocol was approved by the regional ethics committee (Ethics Committee of the RWTH Aachen University Faculty of Medicine, Aachen, Germany; EK163/07) and experiments were carried out in accordance to the principles of the Declaration of Helsinki. Frozen tissue specimens (approx. 1 g) were homogenized in 2 ml lysis buffer (PBS containing a protease inhibitor cocktail; Roche) on ice using a tissue tearer. Homogenates were centrifuged at 2000×g for 10 min to remove large tissue particles, and centrifuged at 14,000×g for 30 min. The clear supernatants of each sample were further divided into 1 ml aliquots, and stored at −80° C. To measure the content of ITIH5 in human adipose tissue, samples were analyzed using the respective Enzyme-Linked Immunosorbent Assay Duo-Sets (R&D Systems, Minneapolis, Minn). Extinction was measured as recommended by the manufacturer using a FLUOstar OPTIMA microplate reader (BMG LABTECH, Aylesbury, United Kingdom) at 450 nm using the reference value of 540 nm. For proper comparison of the cytokine concentrations, we further evaluated the whole protein content of each sample using the DC Protein Assay (BioRad, Hercules, Calif.) as specified by the manufacturer. The cytokine concentration was then normalized to these results as content of soluble factor in picogram per total amount of protein in milligram.

The correlation between ITIH5 protein concentration and BMI was highly significant.

### EXAMPLE 5: RECOMBINANT HUMAN ITIH5 PROTEIN INHIBITS PROLIFERATION IN HUMANS ASCS

The objective of this experiment was to examine the effect of recombinant human ITIH5 protein on the proliferative activity of human adipose stem cells.

Procedures for isolation of human and murine adipose stem cells followed the same protocols. Adipose tissue was minced with surgical scissors, washed with PBS, and digested in 0.2% collagenase for 45 min at 37° C. The stromal cell fraction was separated by centrifugation at 400×g, and filtered through a 250 nm nylon mesh (neoLab, Heidelberg, Germany). After centrifugation, cells were cultured in proliferation medium (DMEM supplemented with 10% FBS, 0.1% bFGF). Experiments were conducted with cells from P2-P4 seeded at a density of 20,000 per cm². Proliferation was assessed after 7 and 14 days of standard culture by measuring cell numbers using crystal violet staining. Cells were washed with PBS and fixed in isopropyl alcohol for 10 min at RT, followed by washing with PBS containing 0.05% Tween20. Then, cells were stained with 0.1% crystal violet for 20 min. After removal of the crystal violet solution, plates were washed with aqua bidest. Adsorbed dye was washed out in 33% acetic acid during 15 min incubation with gentle agitation. 70 μl per sample were transferred in triplets to an optical plate and absorbance was quantified in a microplate reader (BMG Labtech, Ortenberg, Germany) at 620 nm.

Recombinant human ITIH5 protein (amino acid 1-681, i.e. the sequence N-terminal to the conserved cleavage site “DPHFVV” of ITIH proteins (Himmelfarb et al. Cancer Letters 2004, 204, 69-77)) was expressed in human HEK293T cells as described recently (Rose et al. Cancers 2022, 14(3), https://doi.org/10.3390/cancers14030488). After 2 to 4 weeks of culturing, the supernatant of transfected HEK293T cells was harvested in order to isolate the mature ITIH5 protein (amino acid 17-681 as depicted in SEQ ID NO:8). Based on the principle of affinity chromatography, recombinant ITIH5 protein present in the supernatant of HEK293T cells was purified by using Ni-NTA agarose beads (Qiagen, Hilden, Germany). To remove the imidazol for subsequent functional in vitro analyses, isolated ITIH5 protein was further enriched and rebuffered in PBS via Vivaspin 6 columns (Sartorius Stedim, Göttingen, Germany) according to the manufacturer's instructions. Western blot analyses were used to confirm the purity and amount of ITIH5 protein in different protein batches. Stimulation with recombinant human ITIH5 protein significantly decreased the proliferation of human ASCs.

### EXAMPLE 6: RECOMBINANT HUMAN ITIH5 PROTEIN REDUCES DIFFERENTIATION OF HUMANS ASCS

The objective of this experiment was to examine the effect of recombinant human ITIH5 protein on the capability for adipogenic differentiation in human adipose stem cells.

For adipogenic differentiation, cells were exposed to differentiation medium: high glucose medium supplemented with 2% FBS, 10 μM insulin, 0.28 μM rosiglitazone. The media were replaced every 2 to 3 days for 21 days. Adipogenic differentiation was assessed by OilRed O staining. ASCs were PFA fixed, and stained with 0.2% OilRed O solution. Adsorbed dye was washed out with isopropanol. Absorbance was measured in triplets at 540 nm.

While the genetic elimination of the ITIH5 gene significantly increases the adipogenic differentiation of the ASCs (see Example 3), stimulation with the recombinant human ITIH5 protein significantly reduced the adipogenic differentiation of human ASCs.

### EXAMPLE 7: RECOMBINANT HUMAN ITIH5 PROTEIN REDUCES INFLAMMATORY ACTIVITY OF HUMANS ASCS

The objective of this experiment was to examine the effect of recombinant human ITIH5 protein on the inflammatory response of human adipose stem cells.

Background: Increased adiposity is closely associated with a systemic inflammatory state primarily mediated by the increased secretion of pro-inflammatory cytokines, including TNF-α and IL-1β mainly released by M1 macrophages, as well as TL-6 and MCP-1 secreted also by ASCs and mature adipocytes. This chronic inflammatory response is a key driver of the metabolic syndrome, and it increases proportionally to the expansion of adipose tissue.

Material and Methods: In cell culture experiments, isolated ASCs were stimulated with inflammatory mediators diluted in standard medium (1 ng/ml IL-1β and 1 ng/ml TNF-α) and with rITIH5 for 48 h. The cell supernatant was analyzed for IL-6 and MCP-1 release. To measure the amount of inflammatory cytokines, samples were analyzed by ELISA.

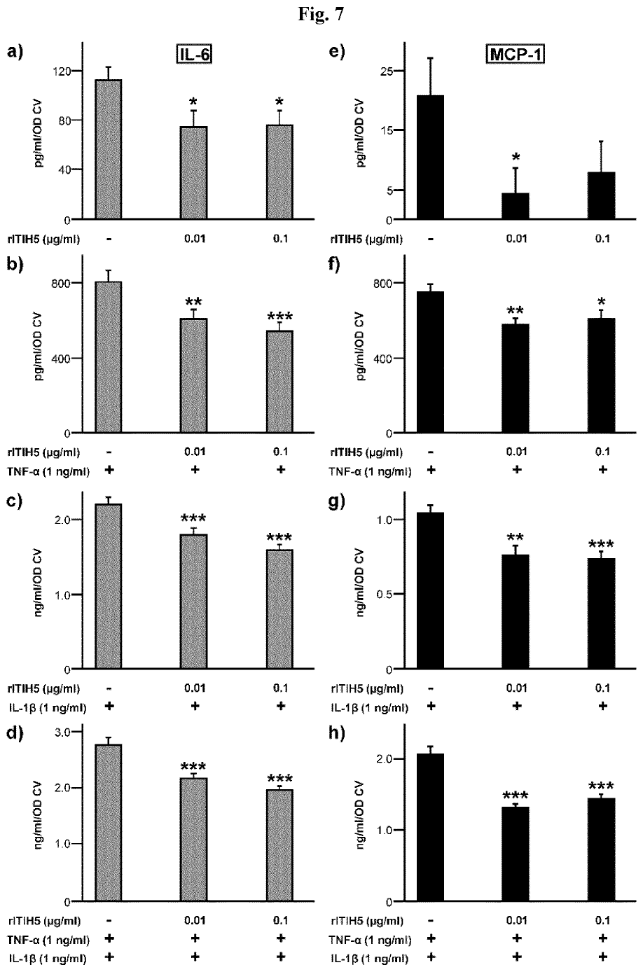

Results: ASCs subjected to an artificial inflammatory environment, mimicked by the presence of TNF-α and IL-1β, increased the release of the pro-inflammatory cytokines IL-6 and MCP-1 as measured by ELISA (FIG. 7). IL-6 was secreted regularly by ASCs at higher concentrations than MCP-1 (FIGS. 7a&e). Each inflammatory mediator increased the production of the investigated cytokines. Low as well as high level of rITIH5 protein decreased significantly the secretion of IL-6 at about 25-30% (p≤0.05, FIG. 7a). A similar effect also occurred when the cells were stimulated with TNF-α (p≤0.01, FIG. 7b) and with IL-1β (p≤0.001, FIG. 7c), or both (p≤0.001, FIG. 7d). Recombinant ITIH5 protein also inhibited the release of MCP-1 (FIG. 7e). However, pairwise comparison detected significant differences due to a stronger decrease by 0.01 μg/ml of rITIH5 protein (p=0.04). Its inhibitory effect was approximately 20% when ASCs were exposed to TNF-α (FIG. 7f), which was significant (p=0.004 for 0.01 μg/ml; p=0.03 for 0.1 μg/ml). After IL-1β stimulation, rITIH5 protein decreased MCP-1 release at about 25% (FIG. 7g), which was significant (p=0.002 for 0.01 μg/ml; p≤0.001 for 0.1 μg/ml). The strongest reduction of MCP-1 secretion was observed when ASCs were co-stimulated with TNF-α and IL-1β ranging at 30% (p≤0.001, FIG. 7h).

1: An isolated or purified ITIH5 polypeptide having the biological activity of a negative regulator of adipogenesis for use in the treatment of obesity in a subject, wherein said treatment comprises reducing weight or reducing weight gain, wherein the ITIH5 polypeptide is to be administered to said subject in an amount therapeutically effective to reduce weight or weight gain, and wherein said ITIH5 polypeptide comprises or consists of a peptide selected from the group consisting of:
a) SEQ ID NO:2, SEQ ID NO:3, SEQ ID NO:4, SEQ ID NO:5, SEQ ID NO:6, SEQ ID NO:7 and SEQ ID NO:8,
b) a biologically active fragment, analog, variant, derivative or elongation of a),
c) a biologically active analog, fragment, variant or derivative of a) having at least 90% identity to SEQ ID NO:2, SEQ ID NO:3, SEQ ID NO:4, SEQ ID NO:5, SEQ ID NO:6, SEQ ID NO:7 and SEQ ID NO:8,
d) a polypeptide encoded by the polynucleotide set out in SEQ ID NO:1,
e) a biologically active fragment, analog, variant, derivative or elongation of d)
f) a polypeptide encoded by a polynucleotide that hybridizes to the polynucleotide set out in SEQ ID NO:9 under moderately stringent hybridization conditions.

2: The isolated or purified ITIH5 polypeptide according to claim 1, wherein the biological activity of a negative regulator of adipogenesis is defined by at least one of the following biological effects:
a. inhibiting the insulin-induced adipogenic differentiation of adipose stem cells (ASCs) into mature adipocytes, and/or
b. inhibiting proliferation of ASCs.

3: The isolated or purified ITIH5 polypeptide according to claim 1, wherein said reducing weight or reducing weight gain comprises at least one of the following:
c. reducing the number of adipose stem/stroma cells, preadipocytes, adipocytes,
d. reducing differentiation, dysregulated differentiation, and maturation of adipose stem/stroma cells and preadipocytes into mature adipocytes,
e. reducing the development, enlargement, and fusion of fat vacuoles and supersized lipid droplets,
f. reducing the amount of adipose tissue,
g. inhibiting dysfunction of adipose tissue,
h. reducing the risk for impaired insulin sensitivity and systemic metabolic deterioration in obese state, e.g., type 2 diabetes, non-alcoholic fatty liver disease, dyslipidemia, hypertension, lipotoxicity.

4: The isolated or purified ITIH5 polypeptide according to claim 1, wherein the subject to be treated is a mammal and preferable a human subject.

5: The isolated or purified ITIH5 polypeptide according to claim 1, wherein the subject to be treated is an obese or overweight subject.

6: The isolated or purified ITIH5 polypeptide according to claim 1, wherein the obesity related attendant symptoms are reduced or prevented, which include inflammation, cardiovascular disease, hypertension, dyslipidemia, heart failure, atherosclerosis, coronary artery disease, stroke, diabetes mellitus type 2, prediabetes, insulin resistance, insulin resistance syndrome (also known as Syndrome X), gall bladder disease, osteoarthritis, sleep apnoea, liver steatosis, obesity-associated liver steatosis, liver inflammation, liver fibrosis, diabetes, obesity-associated fat accretion, obesity-related cancer, infections, varicose veins, acanthosis nigricans, eczema, exercise intolerance, hypertension hypercholesterolemia, cholelithiasis, and decreased life expectancy.

7: The isolated or purified ITIH5 polypeptide according to claim 1, wherein the subject does not has diabetes.

8: The isolated or purified ITIH5 polypeptide according to claim 1, wherein the route of administration of the ITIH5 polypeptide is intravenous, intraperitoneal, intramuscular, subcutaneous, topical, nasal or pulmonary inhalation.

9: A pharmaceutical composition comprising the isolated or purified ITIH5 polypeptide according to claim 1, and at least one pharmaceutically acceptable excipient.

10: A method for identifying epitopes of the ITIH5 protein that reduce weight or reduce weight gain of a subject by modulation of the ITIH5-adipogenic pathway, comprising:
a. synthesizing small peptides with a length of 20 to 40 amino acids covering the whole ITIH5 protein or an adipogenesis-active fragment thereof in an overlapping manner;
b. contacting individual small peptides separately to in vitro cultured adipose stem cells and further incubating the ASCs a preselected time of cell culture under conditions that allow ASC differentiation;
c. determining the ASC differentiation after a preselected time of cell culture by staining of the cells with a fat-soluble dye that stains neutral triglycerides and lipids;
d. selection of small peptides inhibiting ASC differentiation as epitopes of ITIH5 that are capable of reducing weight or reduce weight gain of a subject by modulation of the ITIH5-adipogenic pathway.

11: A method for identifying epitopes of the ITIH5 protein that reduce weight or reduce weight gain of a subject by modulation of the ITIH5-adipogenic pathway, comprising:
a. synthesizing small peptides with a length of 20 to 40 amino acids covering the whole ITIH5 protein or an adipogenesis-active fragment thereof in an overlapping manner;
b. contacting individual small peptides separately to in vitro cultured adipose stem cells and further incubating the ASCs a preselected time of cell culture under conditions that allow ASC proliferation;
c. determining the ASC proliferation by analysing the cells in a cell viability assay such as PrestoBlue® using a detection platform including fluorescence microscopy, flow cytometry, and microplate readers;
d. selection of peptides inhibiting ASC proliferation as epitopes of ITIH5 that are capable of reducing weight or reduce weight gain of a subject by modulation of the ITIH5-adipogenic pathway.

12: A method for identifying synthetic peptides of the ITIH5 protein that reduce weight or reduce weight gain of a subject by modulation of the ITIH5-adipogenic pathway, by selecting a bioactive ITIH5 epitope as identified by the method of claim 10.

13: A method for identifying mimetic small molecules that reduce weight or reduce weight gain of a subject by modulation of the ITIH5-adipogenic pathway, comprising:
a. providing a library of small organic compounds;
b. providing adipocyte precursor cells (APCs) and transfecting them with an expression vector leading to ITIH5 overexpression,
c. selecting single clones from the cells transfected in step b. showing ITIH5 overexpression,
d. providing the adipocyte precursor cells (APCs) as chosen in step b. and transfecting with the expression vector of step. b. not containing the ITIH5 expression construct (control vector) leading to control APCs,
e. selecting single clones from the cells transfected in step d., not exhibiting ITIH5 overexpression (control APCs);
f. in vitro-cultivating at least one clone of the ITIH5-overexpressing APCs and at least one clone of the control APCs and contacting cells from each clone individually with the same small organic compounds of the library provided in step a.,
g. analysing the proliferation or the fat cell differentiation of the clones subjected to the small organic compounds of step f., whereas a ITIH mimicking compound leads to a reduction in APC proliferation or fat cell differentiation in the control APC but not in the ITIH5-overexpressing APCs.
